(19) United States
(12) Patent Application Publication
(10) Pub. No.: US 2007/0074325 A1
(43) Pub. Date: Apr. 5, 2007

(54) Hand-encircling band

(76) Inventor: Gregory K. Rogers, The Colony, TX (US)

Correspondence Address: Stephen R. Greiner, Esquire;GREINER LAW OFFICES, P.C., Suite 110, 6701 Democracy Blvd., Bethesda, MD, 20817, US

(21) Appl. No.: 11/239,296

(22) Filed: Sep. 30, 2005

(51) Int. Cl. A61F 9/00

(52) U.S. Cl. 2/12

(57) ABSTRACT

A hand-encircling band including a strap having a pad, for positioning in the palm of a hand, and a strip of webbing affixed to one end of the pad and extending therefrom. A ring is affixed to the end of the pad remote from the strip of webbing. A first fastening portion is secured to the top of the strip of webbing adjacent the pad. A second fastening portion, adapted to mate with, and releasably adhere to, the first fastening portion, is secured to the top of the strip of webbing adjacent the free end thereof.

### FIELD OF THE INVENTION

The present invention relates generally to apparel and, more particularly to, hand or arm coverings.

### BACKGROUND OF THE INVENTION

Mastering ball handling and shooting skills is fundamental to becoming an excellent basketball player. In an attempt to accomplish this, many players practice for hours on end to correct deficiencies in form and timing. Nonetheless, some highly motivated players never reach their full potential since they never learn how to properly grip a basketball.

To overcome this problem, some have proposed apparatus for wear on the hand that, at all times, properly distances a ball from the palm of a player. This distance requires a player to properly grasp a ball with his fingertips and properly curve his fingers for dribbling, passing and shooting. Prolonged wear of such apparatus can build “muscle memory” to overcome longstanding “palming” tendencies thereby permitting the player to better handle a basketball when the apparatus is not employed.

The known apparatus for promoting a better grip upon a basketball presents drawbacks to a player. For example, many of these products hold the hand and fingers so stiffly that they inhibit finger movement and the development of a “feel” for the ball. Others almost fully cover the hand making such too hot to wear for prolonged training periods. Still others are difficult to put on and take off, making their use uncomfortable and burdensome. For these reasons, apparatus of the sort noted have seen only limited use and commercial development.

### SUMMARY OF THE INVENTION

In light of the problems associated with the known apparatus for teaching proper ball handling techniques to a basketball player, it is a principal object of the invention to provide a hand-encircling band that separates a basketball from the palm of a hand of a player yet permits the fingers of the player to easily move to a proper position for dribbling, passing and shooting. Thus, “muscle memory” is acquired far more rapidly through use of the band than with any other apparatus.

It is a further object of the invention to provide a hand-encircling band of the type described that is adjustable in size to accommodate players with differently sized hands. The adjustment can be accomplished without tools of any sort or prolonged training. The band is, in fact, intuitive to use.

Still another object of the invention is to provide a hand-encircling band that is of utility to players of many sports that involve the throwing and gripping of items like balls, racquets and bats. So, it is believed that a football quarterback will be able to strengthen his grip on a football, and pass more accurately as a result, if he uses the band claimed below. A baseball pitcher might expect similar results. Confidence and game-day aggressiveness are promoted by use of the band.

It is another object of the invention to provide workmen and laborers with a hand-encircling band that can dampen vibrations transmitted up his arms by impact wrenches and other power tools including: saws wrenches, bolt cutters, nail guns, hammers, lawn mowers, and string trimmers.

It is an additional object of the invention to provide a hand-encircling band that can slow the onset of repetitive stress disorders like carpel tunnel syndrome as well as reduce the affects of the repetitive stress disorders that already bother a user. While tending to promote a proper grip upon objects of various sorts, the band can minimize pain in a user. In some cases, use of the band can safely obviate the need for wrist surgery.

It is an object of the invention to provide improved elements and arrangements thereof in a hand-encircling band for the purposes described which is lightweight in construction, inexpensive to manufacture, and dependable in use.

Briefly, the hand-encircling band in accordance with this invention achieves the intended objects by featuring a strap including a pad for positioning in the palm of a hand and a strip of webbing extending from the pad. A ring is affixed to the pad remote from the strip of webbing. A first fastening portion is sewn to the top of the strip of webbing adjacent the pad and has a dense mat of small uncut loops. A second fastening portion, adapted to mate with, and releasably adhere to, the first fastening portion, is secured to the top of the strip of webbing adjacent the free end thereof. The second fastening portion has a number of hooks that are releasably fastenable with the loops of the first fastening portion.

The foregoing and other objects, features and advantages of the present invention will become readily apparent upon further review of the following detailed description of the preferred embodiment as illustrated in the accompanying drawings.

### BRIEF DESCRIPTION OF THE DRAWINGS

The present invention may be more readily described with reference to the accompanying drawings, in which:

FIG. 1 is a top view of a hand-encircling band in accordance with the present invention.

FIG. 2 is a bottom view of the hand-encircling band of FIG. 1.

FIG. 3 is a right side view of the hand-encircling band.

FIG. 4 is a left side view of the hand-encircling band.

FIG. 5 is a perspective view of the hand-encircling band upon the hand of a user.

FIG. 6 is a cross-sectional view taken along line 6-6 of FIG. 1.

FIG. 7 is a front view of the hand-encircling band, and

FIG. 8 is a back view of the hand-encircling band.

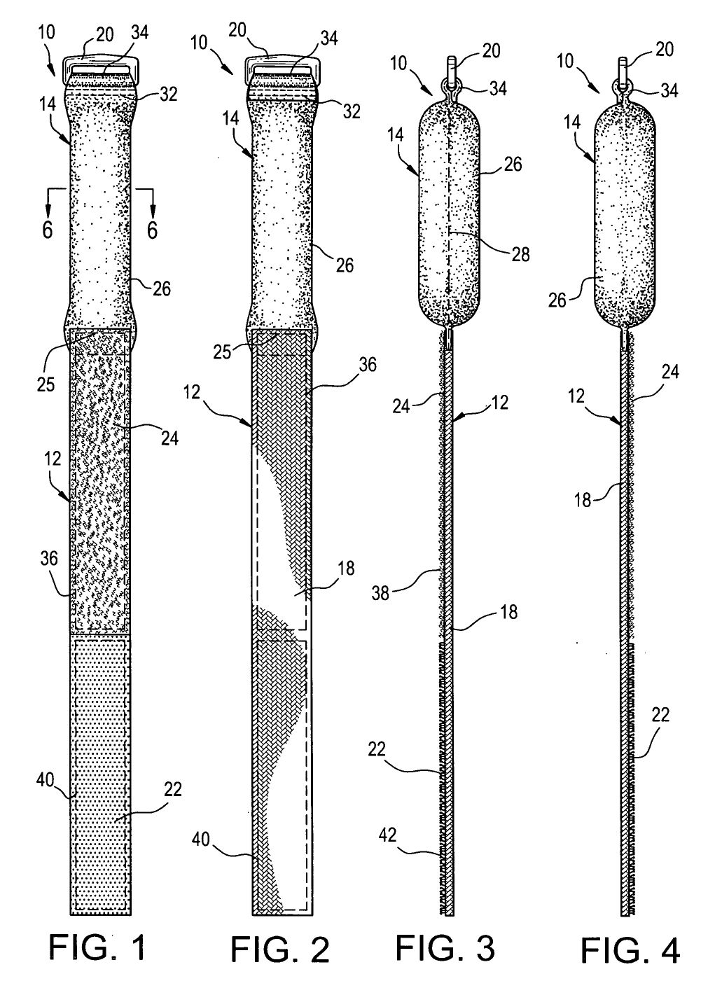

Similar reference characters denote corresponding features consistently throughout the accompanying drawings.

### DETAILED DESCRIPTION OF THE PREFERRED EMBODIMENT

Referring now to the FIGS., a hand-encircling band in accordance with the present invention is shown at 10. Band 10 includes a strap 12 having a pad 14, for positioning in the palm of a hand 16, to the inner end of which is joined a strip of webbing 18 for holding pad 14 in hand 16. The outer end of pad 14 carries a ring 20 that is sized to receive the outer, free end of webbing 18. Affixed to the top of webbing 18 are cooperating hook-and-loop fastening portions 22 and 24 that are utilized to selectively retain webbing 18 in engaging with ring 20 and fix the effective diameter of band 10.

Pad 14 is made by folding a rectangular piece of fabric 26 upon itself and stitching a seam 28 initially across one end of the overlapping parts and, then, along the length of the overlapping parts. Next, the sewn fabric 26 is reversed to hide seam 28 and to provide a flaccid tube into which batting 30 is pressed. After filling the tube of fabric 26 with batting 30, the open end thereof is extended through ring 20, folded back and stitched to itself with a seam 32. Seam 32 prevents the loss of batting from the tube of fabric 26 and forms a loop 34 that retains ring 20. Pad 14 is about 4.75 inches (12.1 cm) long and 1 inch (2.54 cm) in diameter along the majority of its length.

Webbing 18 is a strip of woven nylon or other inelastic material that as attached to the end of pad 14 that lacks loop 34 by means of a sewn seam 25. Nylon, for example, is waterproof and flexible enough to conform to the contours of hand 16. Webbing 18 formed of nylon also has a high coefficient of friction so that it will not readily slide upon hand 16 if wetted. Webbing 16 is about 10.5 inches (26.7 cm) long, 1 inch (2.54 cm) wide, and 0.0625 inches (1.6 mm) thick.

Ring 20 is formed of plastic. Ring 20 extends about 0.625 inch (1.6 cm) in the direction parallel to the longitudinal axis of strap 12 and extends about 1.375 inches (3.49 cm) in the direction transverse to the longitudinal axis of strap 12. Ring 20 is about 0.0625 inches (1.6 mm) thick. Although ring 20 is provided with an O-shaped configuration, it could also be D-shaped if desired.

Fastening portion 24 is secured to the top of webbing 18 by means of a peripheral seam 36. As shown, fastening portion 24 extends from the inner end of webbing 18 adjacent pad 14 to the midpoint of webbing 18. Fastening portion 24 comprises a strip of VELCRO pile material including a dense mat of small, uncut loops 38 formed of thread. Fastening portion 24 has a width that is slightly less than that of webbing 18 and a length of about 5.25 inches (13.3 cm).

By means of sewn peripheral seam 40, fastening portion 22 is secured to the top of webbing 18. Fastening portion 22 extends from the midpoint of webbing 18 to the free, outer end thereof. Fastening portion 22 comprises a strip of VELCRO hook material having a plurality of transverse lines of hooks 42 spaced along its length. The ends of hooks 42 are turned inwardly so as to catch in loops 38 of fastening portion 24 when fastening portions 22 and 24 are pressed together.

Fastening portion 22 has a width that is slightly less than that of webbing 18 and a length of about 5.25 inches (13.3 cm). Because fastening portions 22 and 24 are the same length, fastening portion 22 can be fastened at various points along the length of fastening portion 24 thereby permitting band 10 to be adjustably fastened around hands 16 of different sizes. Thus, band 10 can accommodate users having either large or small hands.

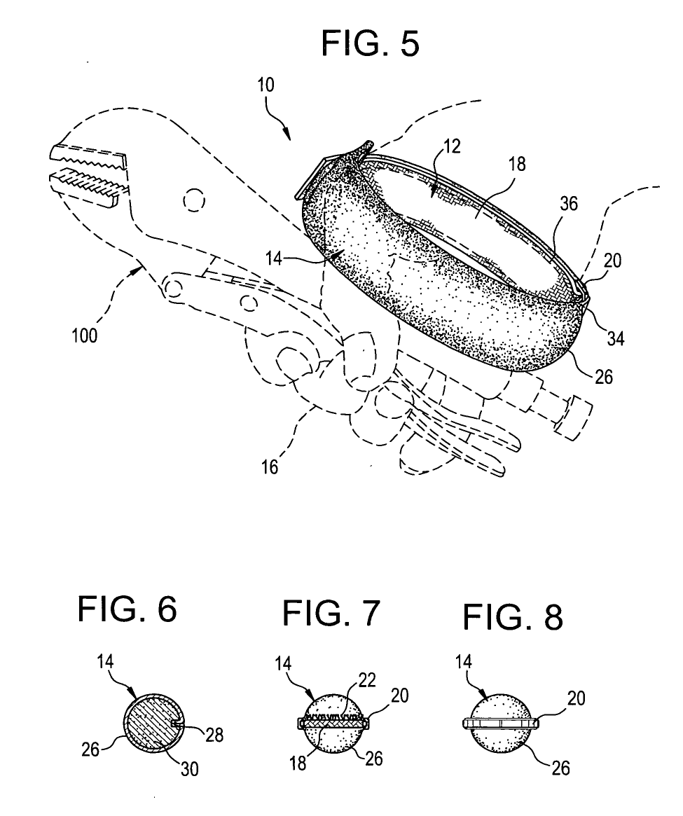

Using band 10 is straightforward. First, pad 14 is positioned in the palm of hand 16 with ring 20 being located remote from the thumb of hand 16 and fastening portions 22 and 24 being faced upwardly from the palm. Then, the free end of webbing 18 is drawn through ring 20 and pulled to the desired degree of tightness. Next, webbing 18 is folded backwardly upon itself so that fastening portion 22 faces fastening portion 24 adjacent the back of hand 16. Now, by firmly pressing fastening portion 22 against fastening portion 24, hooks 42 are caused to enter and grasp loops 34 thereby coupling together the overlapping fastening portions 22 and 24. A user can dribble and shoot a basketball or perform other tasks (like tightening bolts with wrench 100 shown in FIG.5) with band 10 in place. Pad 14 prevents objects, like a basketball, from contacting the palm of hand 16 and promotes the development of “muscle memory” that will ensure that fingers are properly arched when handling a basketball.

Release of interlocked fastening portions 22 and 24 is effected by grasping the free end of webbing 18 and pulling generally outwardly on it. Continued pulling causes hooks 42 and loops 38 to disengage. The transverse line of disengagement progresses lengthwise of the interlocked fastening portions 22 and 24 so that they will separate smoothly. Band 10 can now be easily stored and transported in a gym bag or toolbox in a flat, ready-to-use condition. If soiled, band 10 can be washed in soap and water.

While the invention has been described with a high degree of particularity, it will be appreciated by those skilled in the art that modifications may be made to it. Therefore, it is to be understood that the present invention is not limited to the sole embodiment described above, but encompasses any and all embodiments within the scope of the following claims.

I claim:

1. A hand-encircling band, comprising:
a strap including:
a pad, for positioning in the palm of a hand, having a first end and a second end; and,
a strip of webbing being affixed to said first end of said pad and having a free end remote from said pad;
a ring being affixed to said second end of said pad;
a first fastening portion being secured to the top of said strip of webbing adjacent said pad; and,
a second fastening portion, adapted to mate with, and releasably adhere to, said first fastening portion, being secured to the top of said strip of webbing adjacent said free end thereof.

2. The hand-encircling band of claim 1 wherein said first fastening portion and said second fastening portion are cooperating hook-and-loop fasteners.

3. A hand encircling band, comprising:
a strap including:
a pad, for positioning in the palm of a hand, having a fabric tube stuffed with a batting with said tube having a first end and a second end; and,
a strip of webbing being affixed to said first end of said tube and having a free end remote from said tube;
a ring being affixed to said second end of said tube;
a first fastening portion being secured to the top of said strip of webbing adjacent said tube, said first fastening portion having a dense mat of small uncut loops; and,
a second fastening portion, adapted to mate with, and releasably adhere to, said first fastening portion, being secured to the top of said strip of webbing adjacent said free end thereof, said second fastening portion having a plurality of hooks releasably fastenable with said loops of said first fastening portion.
